(19) United States
(12) Patent Application Publication
(10) Pub. No.: US 2026/0108322 A1
(43) Pub. Date: Apr. 23, 2026

(54) TRACKABLE PET TOYS

(71) Applicant: CARKEN CO., LLC, Mesa, AZ (US)

(72) Inventor: Mark Aaron Carter, San Tan Valley, AZ (US)

(21) Appl. No.: 19/428,474

(22) Filed: Dec. 22, 2025

Related U.S. Application Data
(63) Continuation-in-part of application No. 18/766,434, filed on Jul. 8, 2024.
(60) Provisional application No. 63/512,247, filed on Jul. 6, 2023.

(51) Int. Cl. A61B 90/00; A01K 15/02

(52) U.S. Cl. CPC A61B 90/39; A01K 15/026; A61B 2090/3966; A61B 2503/40

(57) ABSTRACT

A trackable pet toy is disclosed. The pet toy is typically chewable and includes at least one radiopaque component retained by a body of the toy such that, if at least a portion of the toy is ingested by an animal, the ingested portion is detectable using radiographic imaging. A trackable pet toy is disclosed herein. The trackable pet toy has a radiopaque material configured for detection using medical imaging within an animal's digestive system.

### RELATED APPLICATIONS

This application is a continuation-in-part application of U.S. application Ser. No. 18/766,434, filed Jul. 8, 2024, titled “Trackable Pet Toys,” which application claims the benefit of U.S. Provisional Patent Application No. 63/512,247, filed Jul. 6, 2023, titled “Trackable Pet Toys,” the entirety of the disclosures of which are hereby incorporated by this reference.

### TECHNICAL FIELD

This document relates to pet toys, and more specifically to pet toys which, if ingested by a pet, are visible by medical imaging.

### BACKGROUND

Pet toys provide entertainment and stimulation for animals, but they can also pose risks to their health. One significant risk is the potential for pet toys to cause obstructions in the digestive tract. When fragments of these toys are ingested, they may become lodged and lead to serious complications and sometimes death. Thus, it is important to determine whether blockage has occurred and its exact location within the digestive tract.

Imaging techniques may be used to determine whether and where a blockage has occurred. However, depending on the material of the pet toy ingested, standard imaging methods may not be sufficient to answer these questions. For example, toys made from conventional fabric or foam can be hard to see using standard imaging methods. In addition, when the toy is ripped or torn into small, irregularly shaped pieces, these pieces can be hard to identify, in particular when they are obscured by surrounding tissues.

### SUMMARY OF THE INVENTION

According to some embodiments, the present disclosure relates to a chewable pet toy comprising a body defining an exterior layer and an interior volume, and a radiopaque component retained by the body, wherein the radiopaque component is disposed in a portion less than all of at least one of the exterior layer or the interior volume and wherein the radiopaque component is detectable by radiographic imaging when located within a digestive tract of an animal.

Particular embodiments may comprise one or more of the following features. The radiopaque component may comprise a radiopaque filament incorporated into a textile of the exterior layer. The textile may comprise a woven fabric having a thread count, and a fraction of radiopaque threads in the thread count may be from 1 in 3 to 1 in 2000. The fraction of radiopaque threads may be from 1 in 10 to 1 in 300. The radiopaque component may comprise a radiopaque coating bonded to fibers of the exterior layer. The radiopaque coating may comprise a polymer matrix loaded with a radiopaque filler at a loading of from 10 wt % to 70 wt %. The chewable pet toy may further comprise stuffing material disposed in the interior volume, wherein the stuffing material includes radiopaque stuffing elements and non-radiopaque stuffing elements. The radiopaque stuffing elements may comprise from 0.5% to 50% of the stuffing elements by count. The radiopaque stuffing elements may have a characteristic dimension from 1 mm to 20 mm, and the pet toy may comprise at least 25 radiopaque stuffing elements. The radiopaque component may be distributed across a plurality of locations of the body including at least two spatially separated exterior zones.

According to some embodiments, the present disclosure relates to a trackable pet toy comprising a woven fabric forming an external surface of a body, the woven fabric having a thread count, stuffing disposed within an interior volume of the body, wherein a minority of the threads comprise radiopaque thread material, wherein a minority of stuffing elements comprise radiopaque stuffing elements, and wherein the radiopaque thread material and the radiopaque stuffing elements are water insoluble and retained by the trackable pet toy under washing and saliva exposure, such that the trackable pet toy is x-ray detectable after ingestion by an animal.

Particular embodiments may comprise one or more of the following features. The minority of the threads may comprise from 1 in 5 to 1 in 500 threads of the thread count. The radiopaque thread material may include a core-sheath filament having a radiopaque core encapsulated by a non-radiopaque sheath. Radiopaque material may be disposed along from 10% to 80% of a total seam length of the woven fabric. Radiopaque material may be disposed in from 2 to 12 discrete exterior zones of the external surface. The radiopaque stuffing elements may comprise foam pieces and/or fiberfill fibers including tungsten and/or barium sulfate. The radiopaque stuffing elements may comprise from 1% to 30% of the stuffing elements by mass. The trackable pet toy may further comprise a noise-emitting component disposed within the interior volume, wherein at least a portion of the noise-emitting component is radiopaque.

According to some embodiments, the present disclosure relates to a method of manufacturing a trackable pet toy comprising incorporating a radiopaque filler into at least one of (i) threads used to form an exterior layer of a pet toy, (ii) stuffing elements used to fill an interior volume of the pet toy, or (iii) a polymeric coating applied to a textile component of the pet toy, assembling the pet toy so that the radiopaque filler is retained by the pet toy and disposed in a portion less than all of at least one of the exterior layer or the interior volume, and confirming radiographic detectability of the pet toy via radiographic imaging.

Particular embodiments may comprise one or more of the following features. Incorporating the radiopaque filler may comprise dispersing barium sulfate and/or tungsten in a polymer matrix and forming the polymer matrix into at least one of a filament, a foam piece, or a coating.

According to some embodiments, the present disclosure relates to a trackable pet toy comprising a woven fabric forming an external surface, the woven fabric having a thread count, wherein at least one and less than all of the threads of the thread count comprise a radiopaque material, and an interior volume inside of the woven fabric, the interior volume comprising a stuffing material, wherein at least a portion of the stuffing material comprises the radiopaque material, wherein the radiopaque material is configured for detection using x-ray imaging within a digestive system of an animal.

Particular embodiments may comprise one or more of the following features. Less than all of the stuffing material may comprise the radiopaque material. 1 in 10 to 1 in 3 threads of the thread count may comprise the radiopaque material. The stuffing material may comprise fiberfill. The fiberfill may comprise a blend of radiopaque and non-radiopaque fibers. The stuffing material may comprise a plurality of foam pieces, wherein the plurality of foam pieces comprises a blend of radiopaque and non-radiopaque foam pieces. The stuffing material may comprise a plurality of pieces of fabric.

According to some embodiments, the present disclosure relates to a trackable pet toy comprising an external surface and an interior volume, wherein at least a portion and less than all of the external surface comprise a radiopaque material, and a stuffing material disposed within the interior volume, wherein at least a portion and less than all of the stuffing material comprises a radiopaque material configured for detection using x-ray imaging within a digestive system of an animal.

Particular embodiments may comprise one or more of the following features. The external surface may be formed of a woven fabric having a thread count, wherein at least one and less than all of the threads of the thread count comprise the radiopaque material. 1 in 10 to 1 in 3 threads of the thread count may comprise the radiopaque material. The stuffing material may comprise fiberfill. The fiberfill may comprise a blend of radiopaque and non-radiopaque fibers. The stuffing material may comprise a plurality of foam pieces, wherein the plurality of foam pieces comprises a blend of radiopaque and non-radiopaque foam pieces. The stuffing material may comprise a plurality of pieces of fabric.

According to some embodiments, the present disclosure relates to a trackable pet toy comprising an external surface and an interior volume, and a stuffing material disposed within the interior volume, wherein at least a portion of the stuffing material comprises a radiopaque material configured for detection using x-ray imaging within a digestive system of an animal.

Particular embodiments may comprise one or more of the following features. Less than all of the stuffing material may comprise the radiopaque material. The stuffing material may comprise fiberfill. The fiberfill may comprise a blend of radiopaque and non-radiopaque fibers. The stuffing material may comprise a plurality of foam pieces, wherein the plurality of foam pieces comprises a blend of radiopaque and non-radiopaque foam pieces. The stuffing material may comprise a plurality of pieces of fabric.

The disclosure concerns a trackable pet toy including a woven fabric, the trackable pet toy having an external surface and an interior volume, wherein at least a portion of the trackable pet toy includes a radiopaque material configured for detection using x-ray imaging within a digestive system of an animal.

In some aspects, the disclosure concerns a trackable pet toy, wherein the animal is a dog and at least a portion of the external surface, at least a portion of the interior volume, or both includes the radiopaque material.

In further aspects, the disclosure concerns a trackable pet toy, wherein at least a portion of the external surface and the interior volume include the radiopaque material.

In some aspects, the disclosure concerns a trackable pet toy, wherein the woven fabric has a thread count, wherein at least one thread of the count includes the radiopaque material.

In additional aspects, the disclosure concerns a trackable pet toy, wherein 1 in 10 to 1 in 300 threads is the radiopaque material.

In some aspects, the disclosure concerns a trackable pet toy, wherein the woven fabric is impregnated with the radiopaque material, the radiopaque material being water insoluble.

In some aspects, the disclosure concerns a trackable pet toy, wherein the trackable pet toy is formed in the shape of an animal including an ear, a leg, a nose, and/or a tail, the ear, leg, nose, and/or tail having at least one thread of the radiopaque material.

In further aspects, the disclosure concerns a trackable pet toy, further including a noise emitting component, wherein at least as portion of the noise emitting component comprises the radiopaque material.

In some aspects, the disclosure concerns a trackable pet toy, wherein the noise emitting component comprises one selected from the group consisting of a squeak device, a plastic film, crinkle paper, and a silent ultrasonic squeaker device.

In some aspects, the disclosure concerns a radiopaque pet toy including an external surface and an interior volume, wherein at least a portion of the external surface or at least a portion of the interior volume includes a radiopaque material configured for detection using medical imaging within a digestive system of an animal.

In further aspects, the disclosure concerns a radiopaque pet toy, wherein one of: at least a portion of the external surface and/or the interior volume includes a woven fabric including the radiopaque material, the radiopaque material being water insoluble.

In some aspects, the disclosure concerns a radiopaque pet toy further including a noise emitting component including the radiopaque material.

In some aspects, the disclosure concerns a radiopaque pet toy, wherein the noise emitting component includes one selected from the group consisting of: a squeak device; a plastic film; crinkle paper, and a silent ultrasonic squeaker device.

In some aspects, the disclosure concerns a radiopaque pet toy, wherein the noise emitting component is disposed in the interior volume.

In additional aspects, the disclosure concerns a radiopaque pet toy, wherein the noise emitting component includes a squeaker device.

In some aspects, the disclosure concerns an x-ray detectable pet toy comprising at least one radiopaque material, the x-ray detectable pet toy having an external surface and an interior volume and including a noise emitting component configured for detection using x-ray imaging within a digestive system of an animal.

In some aspects, the disclosure concerns an x-ray detectable pet toy, wherein the noise emitting component includes a radiopaque material, the noise emitting component being selected from the group consisting of: a squeak device; a plastic film; crinkle paper, and a silent ultrasonic squeaker device, wherein the noise emitting component is disposed between the external surface and the interior volume, or within the interior volume.

According to further aspects, the disclosure concerns a x-ray detectable pet toy, wherein the at least one radiopaque material is a woven fabric having a thread count, wherein at least one thread of the woven fabric includes the radiopaque material.

In some aspects, the disclosure concerns a x-ray detectable pet toy, wherein 1 of 10 to 1 of 200 threads of the woven fabric includes the radiopaque material and the x-ray detectable pet toy is formed in the shape of an animal.

In additional aspects, the disclosure concerns an x-ray detectable pet toy, wherein the radiopaque material includes elements of atomic numbers greater than or equal to 29.

The foregoing and other aspects, features, and advantages will be apparent from the description and drawings, and from the claims.

### BRIEF DESCRIPTION OF THE DRAWINGS

Implementations will hereinafter be described in conjunction with the appended and/or included drawings, where like designations denote like elements.

FIGS. 1 and 2 are representations of trackable pet toys according to some embodiments disclosed herein.

FIGS. 3A and 3B are representations of noise emitting components according to some embodiments disclosed herein.

FIGS. 4A and 4B are embodiments of woven fabrics comprising radiopaque materials.

FIG. 5 is a representation of the digestive system of a dog comprising a portion of a radiopaque pet toy.

FIGS. 6A, 6B and 6C are representations of a trackable pet toy according to some embodiments, illustrating the interior of the pet toy.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The words “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for clarity and understanding and are not meant to limit or restrict the disclosed subject matter or relevant portions of this disclosure. It is to be appreciated that a myriad of additional or alternate examples of varying scope could have been presented but have been omitted for purposes of brevity.

When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises”, mean “including but not limited to”, and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions. The term “plurality”, as used herein, means more than one.

The present disclosure relates to a trackable pet toy that may be used as a chew toy by a dog. While pet toys typically provide numerous benefits to animals, if ingested, they can also pose risks to their health by causing obstructions in the digestive tract. If the toy, or portions of the toy are ingested, they may become lodged in the digestive tract and lead to serious complications requiring surgical intervention, and sometimes death. Thus, it is important to determine whether blockage has occurred in the digestive system, and if so, to determine the location of the pet toy, or the portion of the pet toy, forming the blockage within the digestive system. Imaging techniques may be used to determine whether and where a blockage has occurred. However, depending on the material the pet toy ingested is made from, standard imaging methods may be insufficient to locate or identify the ingested pet toy. For example, toys made from conventional fabric or foam, having inherently low densities, can be hard to differentiate from surrounding tissue using standard imaging methods. In addition, when the toy is ripped or torn into any number of small, irregularly shaped pieces, these pieces can be hard to locate in the digestive tract, in particular when they have a similar density to surrounding tissues and may be obscured by the tissues.

Because even the sturdiest, most durable pet toys are frequently still chewed up, torn apart, and ingested, it is desirable for the well-being of the pet to make the pet toy capable of being located within the pet's digestive tract once ingested to prevent or remove blockage within the digestive tract. Thus, as will be described in more detail below, the pet toy is designed to be visible, or more visible, using standard imaging techniques. This allows veterinarians to use existing, in-office equipment to determine whether there is a blockage in the intestinal tract and thus act quickly to remedy the problem without taking unnecessary actions, or if more serious interventions are needed, proceeding accordingly in a timely manner.

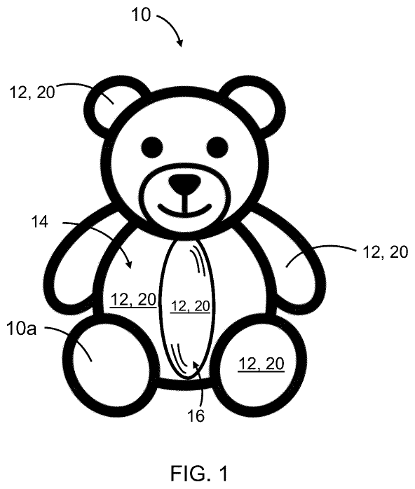

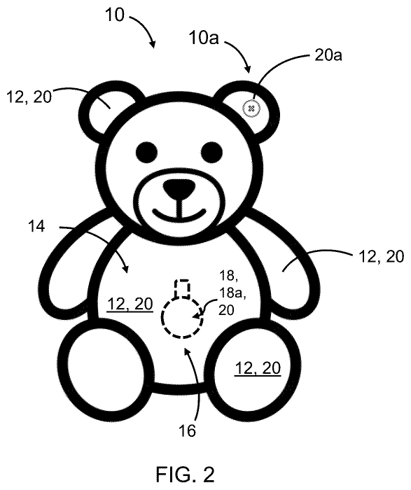

Disclosed is a trackable pet toy 10, also referred to herein as an x-ray detectable pet toy or a radiopaque pet toy, the trackable pet toy 10 comprising a woven fabric 12, the trackable pet toy 10 having an external surface 14 and an interior volume 16. The trackable pet toy 10 and at least a portion 10a of the trackable pet toy 10, may comprise a radiopaque material 20 configured for detection using medical imaging within a digestive system 32 of an animal 30. In some embodiments, such as in FIGS. 1 and 2, the radiopaque material 20 may comprise part or all of the external surface 14. In further embodiments, the radiopaque material 20 may be disposed in the interior volume 16. In some embodiments as depicted in FIG. 1, the trackable pet toy 10 may comprise an interior volume 16 comprising a pocket or compartment which may have an object comprising the radiopaque material 20 disposed therein. In yet further embodiments according to FIG. 1, the radiopaque material 20 may comprise part or all of an internal surface of the interior volume 16. In those embodiments where the radiopaque material 20 comprises part or all the internal surface, the trackable pet toy 10 may be cleaned or washed without exposure or degradation of the radiopaque material 20 during cleaning. In some embodiments as depicted in FIG. 2, the trackable pet toy 10 may comprise an interior volume 16 comprising the radiopaque material 20 and surrounded by the external surface 14. According to some embodiments, the radiopaque material 20 may be water insoluble.

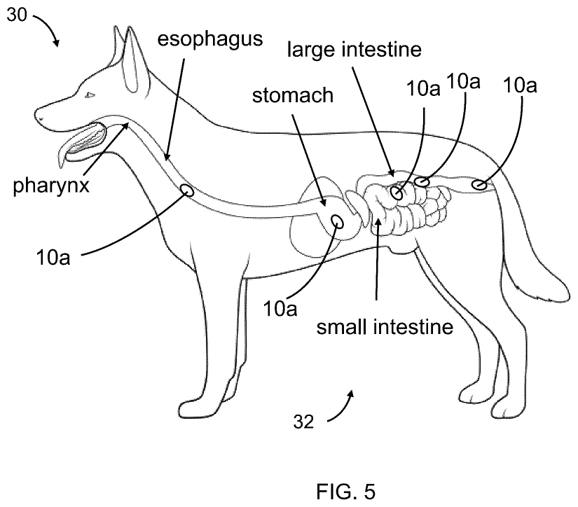

The disclosure may relate to any animal or pet, and in one embodiment, the animal is a dog. Typical medical imaging methods which may be used to detect the trackable pet toy 10, or a portion 10a of the trackable pet toy 10 in the digestive tract 32 of an animal 30 may comprise x-ray, MRI, CT, x-ray fluoroscopy, ultrasound, radiography and other methods as known in the art. As depicted in FIG. 5, the digestive system 32 of an animal 30 comprises several different anatomical parts, and the trackable pet toy 10 is able to be detected using the medical imaging techniques as disclosed herein within any of the parts of the anatomy as shown.

### Definitions

As used herein, “chewable” means that a pet toy is made, adapted, or intended to be chewed by an animal during play (e.g., biting, gnawing, compressive loading) and includes toys specifically designed for chewing as well as toys intended to be held, carried, or otherwise manipulated in the animal's mouth even if not expressly intended for chewing. The term “chewable” is purpose-based and does not require any particular material, construction, geometry, or hardness unless expressly stated. Without limitation, a chewable pet toy may be formed from flexible or resilient materials such as woven or knitted textiles, elastomers, rope, foam, polymer composites, and combinations thereof, and may include structures incorporating layered fabrics, coatings, marker strips, or internal components (e.g., squeakers or crinkle elements). These examples are illustrative and non-limiting.

As used herein, “radiopaque” and “x-ray detectable” mean that, under radiographic imaging parameters used in routine veterinary practice, the radiopaque component attenuates x-rays sufficiently to be visually distinguishable from surrounding soft tissue in at least one radiographic image.

As used herein, “radiographic imaging” includes at least x-ray radiography, x-ray fluoroscopy, and computed tomography (CT). As used herein, a “radiographic image” includes any image produced by radiographic imaging. References to “x-ray imaging” should be understood to encompass these radiographic modalities unless the context indicates otherwise.

As used herein, a “radiopaque material” may comprise a polymeric composition (e.g., elastomeric or thermoplastic) loaded with a radiopaque filler (e.g., barium sulfate, bismuth compounds/oxides, tungsten, tantalum, zirconium oxide, or other high-atomic-number materials), a metal or metal alloy, and/or a radiopaque dye or pigment.

As used herein, a “portion” of a pet toy component (e.g., exterior layer, seam, coating, stuffing) may be expressed by one or more of: (i) areal coverage (percent of exterior surface area), (ii) linear coverage (percent of seam length), (iii) count fraction (percent of threads/filaments or discrete elements), and/or (iv) mass or volume fraction, as specified. As used herein, a “minority” means less than 50% by the specified metric.

As used herein, “water insoluble” and “retained” mean that the radiopaque material remains associated with the pet toy component under ordinary washing/cleaning and saliva exposure (e.g., by being encapsulated in a polymer matrix, bonded to fibers, or mechanically trapped), rather than dissolving and dispersing such that radiopaque functionality is substantially lost.

As used herein, “distributed across a plurality of locations” means present at two or more spatially separated locations of the body, such as on opposing sides of the exterior layer and/or within the interior volume, to increase a likelihood that a torn or ingested fragment includes radiopaque material.

In some embodiments, “detectable” may be evaluated by obtaining at least one standard veterinary radiograph of the animal and visually identifying the radiopaque material as distinguishable relative to adjacent soft tissue (and, in some embodiments, relative to non-radiopaque portions of the same toy).

Radiopaque substances, and the radiopaque materials 20 formed therefrom, typically include heavy atoms, such as elements of atomic numbers greater than or equal to 29, comprising calcium, barium, titanium, copper, silver, gold, platinum, tantalum, tungsten, gadolinium, iodine, strontium, lanthanum, yttrium, zirconium, and other high atomic number elements, as well as stainless steel, and other alloys or inorganic materials having high densities such that they would be visible using the medical imaging methods as disclosed. As one example, radiopaque substances may be provided as the inorganic salts of heavy elements, which may be further processed to form the radiopaque materials 20 as described following. Further examples of useful radiopaque substances may include iodine-based contrast agents such as iodixanol, iohexol, and iopamidol; gadolinium-based contrast agents such as gadodiamide, gadopentetate dimeglumine, and gadobenate dimeglumine; bismuth subsalicylate; calcium tungstate; silver; tantalum; platinum; gold; and barium-impregnated polyethylene spheres, among others.

The radiopaque materials 20, comprising the aforementioned radiopaque substances, may be incorporated into a variety of articles according to end use. Some examples of radiopaque materials 20 and articles formed therefrom which may comprise one or more of the aforementioned radiopaque substances include fabrics, dyes, cloth, a filler material, sponge, compressible recyclable material, threads, inorganic materials (based upon the aforementioned heavy atoms), and various radiopaque polymers formed as layers, sheets, strips, beads, or composites, each of which may be impregnated with one or more radiopaque substances. In some embodiments, radiopaque polymers may be provided in solution form for application to the aforementioned materials to form the radiopaque materials 20.

In certain embodiments, the radiopaque material 20 may comprise a polymeric coating, polymeric layer, polymeric tape or strip, polymeric matrix, a plurality of beads, a plurality of threads formed into a yarn, a polymer composite, inorganic materials, and similar materials to those recited, as well as combinations thereof. As illustrated in FIGS. 1 and 2, the trackable pet toys 10 may be manufactured or made from the aforementioned radiopaque materials 20. According to some embodiments, the radiopaque material 20 may be water insoluble such that when the trackable pet toy 10 is chewed, ingested, or partially digested by an animal, such as a dog, the radiopaque material 20 remains as part of the trackable pet toy 10, thereby facilitating its location within the animal's digestive tract 32 over time.

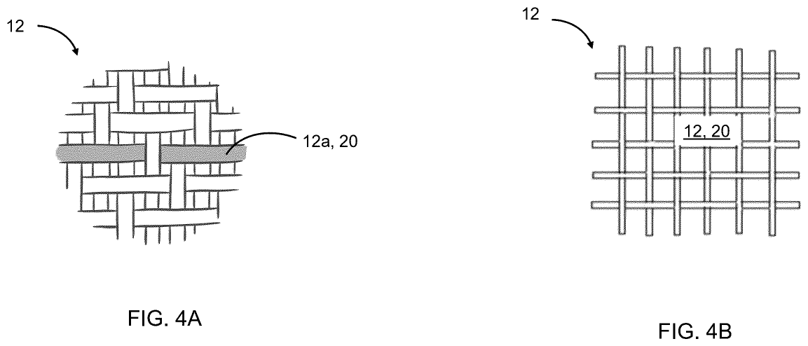

According to some embodiments as shown in FIGS. 1 and 2, at least a portion of the external surface 14, at least a portion of the interior volume 16, or both, may comprise the woven fabric 12 where the woven fabric 12 comprises the radiopaque material 20. In some embodiments, the woven fabric 12 may be made of natural textile materials, or from woven synthetic materials, such as rubbers and polymers. In some embodiments, the trackable pet toy 10 may be made from more than one layer of woven fabric 12 to enhance durability. To make the trackable pet toy 10 more visible using standard imaging techniques, when the trackable pet toy 10, or a portion 10a of the trackable pet toy 10, comprising at least one thread 12a of the radiopaque material 20, has been swallowed by the pet, all or the portion 10a of the pet toy may be manufactured or made with radiopaque materials 20. The trackable pet toy 10 may be formed in the shape of an animal comprising an ear, a leg, a nose, and/or a tail, the ear, leg, nose, and/or tail from a woven fabric 12 having at least one thread 12a of the radiopaque material 20. An exemplary woven fabric 12 comprising the radiopaque material 20 is depicted in FIG. 4A. As shown, the woven fabric 12 may comprise at least one thread 12a, comprising the radiopaque material 20 as disclosed herein. The woven fabric 12 may comprise a thread count similar to that used for other textile materials, such as a thread count of from 100 to 500, where at least one thread of the thread count comprises the radiopaque material 20. Thread count is a measure of the number of threads woven into one square inch of fabric. In some embodiments, 1 in 5 to 1 in 300 threads of the count may comprise the radiopaque material 20.

In certain nonlimiting exemplary embodiments, 1 thread in 10 to 1 thread in 200 threads of the count may comprise the radiopaque material 20, and in further embodiments, 1 thread in 50 to 1 thread in 400 in threads of the count may comprise the radiopaque material 20. In some embodiments, every other thread of the thread count may comprise the radiopaque material 20, and in yet further embodiments, every thread of the thread count may comprise the radiopaque material 20 such that the woven fabric 12 is completely formed from the radiopaque material 20. According to further embodiments, the woven fabric 12 may comprise a rubber material, such as nylon or ballistic nylon, woven using at least one thread 12a which is synthetic and comprises the radiopaque material 20, and additional materials having similar properties, woven in a basket weave pattern with similar thread counts as disclosed herein.

In further nonlimiting embodiments, the exterior layer comprises a woven fabric having a total thread count, and a minority of the threads comprise or include radiopaque material. For example, the fraction of radiopaque threads may be from 1 in 3 to 1 in 2000, optionally from 1 in 5 to 1 in 500, optionally from 1 in 10 to 1 in 300, optionally from 1 in 25 to 1 in 200, and optionally from 1 in 50 to 1 in 150. In some embodiments, selecting a minority fraction of radiopaque threads helps preserve flexibility, hand-feel, and chewability while still providing radiographic detectability.

Additionally or alternatively, a radiopaque portion of the exterior layer may be defined by areal coverage of radiopaque regions on the exterior surface (e.g., by coating, printing, or attachment of a marker strip). By way of example, radiopaque areal coverage may be from about 0.1% to about 80% of the exterior surface area, optionally from about 0.5% to about 50%, optionally from about 1% to about 30%, and optionally from about 2% to about 20%.

In some embodiments, radiopaque material is disposed along seams of the exterior layer. By way of example, radiopaque material may be disposed along about 5% to about 100% of a total seam length of the exterior layer, optionally from about 10% to about 80%, optionally from about 20% to about 60%, and optionally from about 30% to about 50%. In some embodiments, seam-based placement provides radiographic detectability while limiting radiopaque material in high-flex regions of the exterior layer.

In some embodiments, radiopaque stuffing elements have a characteristic dimension from about 0.5 mm to about 30 mm, optionally from about 1 mm to about 20 mm, and optionally from about 2 mm to about 10 mm. In some embodiments, a plurality of radiopaque stuffing elements are provided (e.g., at least 10, at least 25, or at least 50 radiopaque stuffing elements per toy) to increase a likelihood that a removed or ingested fragment includes radiopaque material.

In some embodiments, the interior volume comprises stuffing material including radiopaque stuffing elements and non-radiopaque stuffing elements. The radiopaque stuffing elements may comprise, for example, fiberfill fibers, foam pieces, and/or fabric pieces including radiopaque material. The radiopaque fraction of stuffing elements may be from about 0.1% to about 70% by count, mass, or volume (as specified), optionally from about 0.5% to about 50%, optionally from about 1% to about 30%, optionally from about 2% to about 20%, and optionally from about 5% to about 15%. In some embodiments, the radiopaque stuffing elements have a characteristic dimension of about 0.5 mm to about 30 mm, optionally about 1 mm to about 20 mm, optionally about 2 mm to about 10 mm, and the stuffing includes at least 10, optionally at least 25, optionally at least 50 radiopaque stuffing elements per toy.

In some embodiments, radiopaque material is distributed across a plurality of discrete exterior zones and/or interior locations to increase a likelihood that a torn or ingested fragment of the pet toy includes radiopaque material. For example, radiopaque material may be disposed in 2 to 12 discrete exterior zones (e.g., ears, legs, nose, tail, or other regions), optionally on opposing sides of the exterior layer, and/or in a plurality of interior clusters spaced throughout the interior volume. In some embodiments, radiopaque locations are separated by at most about 10 cm, optionally at most about 5 cm, optionally at most about 3 cm, measured along a surface of the toy or through the interior volume.

In one embodiment, as depicted in FIG. 4B, the radiopaque materials 20 are applied to the woven fabric 12 using one or more of the methods disclosed herein, such that the woven fabric 12 comprises radiopaque materials 20 over a surface and/or throughout. This embodiment provides a means of detecting portions of the fabric within the digestive system of the pet. According to some embodiments, as shown in FIGS. 1 and 2, the trackable pet toy 10 may comprise radiopaque materials 20 impregnated into, or sprayed over, the woven fabric 12 where the radiopaque material 20 is water insoluble and thus will not come off during chewing, ingestion or digestion.

In some embodiments, a radiopaque material 20 may be provided in a wire or thread form, either in elemental form or as an alloy, such as silver, titanium, barium, copper, gold, platinum, tungsten, stainless steel, or tantalum, and alloys thereof, and may be part of the woven fabric 12, comprise individual threads of the woven fabric 12, or may be attached to a thread of the trackable pet toy 10. Such radiopaque material 20, in wire or thread form, may be of much smaller dimension so that it is not necessarily noticeable visually or by touch. The radiopaque material may be attached to the thread at regular intervals or continuously along the length of the thread. These radiopaque threads may then be incorporated into the pet toy in any location on the pet toy, whether in the interior or on the exterior, and in any pattern to increase the likelihood that some radiopaque threads are ingested along with any ingested portions of the pet toy. Additionally, in some embodiments as aforementioned, the thread itself may be formed from the radiopaque material 20. As mentioned above, the radiopaque material 20 may be any material that has a high atomic number and density.

In some embodiments, the radiopaque material 20 comprises threads or fabric of the pet toy that may also be dyed with a dye comprising a radiopaque substance or coated with a radiopaque material 20, such as a polymer coating comprising a radiopaque material 20. Examples of radiopaque substances that could be used as part of a dye include barium sulfate, iodine-based contrast agents such as iohexol or iopamidol, and various bismuth compounds, such as bismuth subsalicylate. The same radiopaque substances could also be used as part of the radiopaque material to coat the thread, though the formulation may be different if a coating is desired rather than a dye. In some embodiments, the threads or fabric of the trackable pet toy may be coated with a radiopaque polymer by submersion in a solution containing radiopaque polymers comprising the aforementioned radiopaque substances. Additionally, radiopaque metals, such as silver, titanium, barium, copper, gold, platinum, tungsten, stainless steel, or tantalum, and alloys thereof, could be used in combination with the radiopaque polymers to coat the threads with a water insoluble radiopaque material 20. In an embodiment, the threads or fabric of the pet toy may be coated with a traditional polymer having low radiopacity, and the aforementioned radiopaque metals disposed therein.

In some embodiments, the pet toy 10 comprises an external surface 14 and an interior volume 16. At least a portion of the external surface 14 may comprise a radiopaque material 20. Similarly, at least a portion of the interior volume 16 may comprise a radiopaque material 20. In some embodiments, less than all of the external surface 14 comprises a radiopaque material 20. The pet toy 10 may comprise a stuffing material 22 disposed within the interior volume 16. In some embodiments, at least a portion and/or less than all of the stuffing material 22 comprises a radiopaque material 20, which is configured for detection using x-ray imaging within a digestive system of an animal.

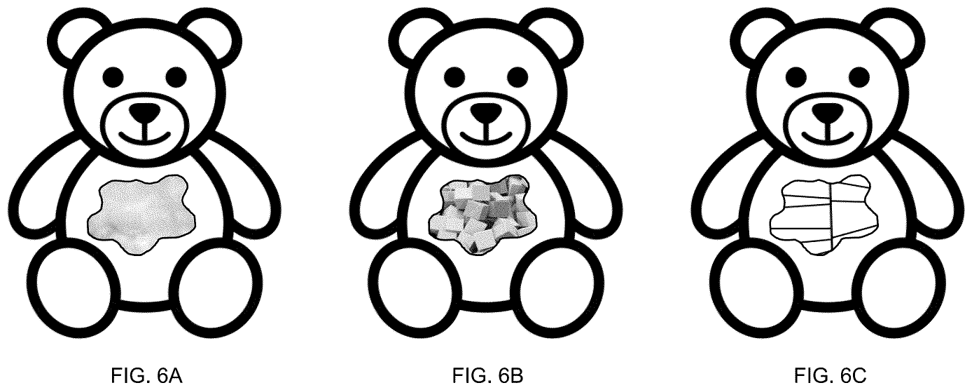

In some embodiments, the stuffing material 22 disposed within the interior volume 16 of the pet toy 10 comprises fiberfill, as shown in FIG. 6A. Fiberfill may include loose, fluffy synthetic or natural fibers, such as polyester, cotton, or blends thereof, commonly used as stuffing in plush toys and pillows. The fiberfill may comprise a blend of radiopaque and non-radiopaque fibers 24, wherein the radiopaque fibers include, for example, fibers impregnated, coated, or manufactured with a radiopaque substance such as barium sulfate, tungsten, titanium, stainless steel, or bismuth oxide. The ratio of radiopaque to total fibers may be selected to achieve a desired level of radiopacity, for example, from 1 in 10 to 1 in 3 fibers being radiopaque. The ratios disclosed below regarding the woven fabric may also be implemented for the ratio of radiopaque to total fibers of the stuffing material 22.

In some embodiments, the stuffing material 22 comprises a plurality of foam pieces 26, as shown in FIG. 6B. The foam pieces 26 may be cubical, spherical, or irregularly shaped, and may be formed from polyurethane, memory foam, latex, or other suitable materials. At least a portion of the foam pieces 26 may comprise a radiopaque material, such as by incorporating radiopaque powder into the foam matrix during manufacturing, or by coating or impregnating the foam pieces with a radiopaque polymer. The stuffing material 22 may also comprise a blend of radiopaque and non-radiopaque foam pieces, with the ratio of radiopaque pieces selected to provide sufficient detectability by x-ray imaging, similar to the ratios of the fibers 24 of the fiberfill discussed above.

In some embodiments, the stuffing material 22 comprises a plurality of pieces of fabric 28, as shown in FIG. 6C. The fabric pieces 28 may be cut or shredded from woven or nonwoven textiles, and at least a portion of the fabric pieces 28 may comprise radiopaque material, such as by including radiopaque threads, coatings, or impregnations. The stuffing material may thus be a blend of radiopaque and non-radiopaque fabric pieces.

The radiopaque aspect of the stuffing material 22 may be implemented in various ways. For fiberfill, radiopaque fibers 24 may be manufactured by incorporating radiopaque substances into the fiber extrusion process, by coating fibers with a radiopaque polymer, or by impregnating fibers with radiopaque particles. For foam pieces 26, radiopaque powder may be mixed into the foam precursor prior to curing, or foam pieces may be coated or impregnated with a radiopaque solution after formation. For fabric pieces 28, radiopaque threads may be woven into the fabric prior to cutting, or the fabric may be coated or impregnated with a radiopaque material before or after being cut into pieces. The stuffing material 22 may be formed by mixing radiopaque and non-radiopaque components to achieve a desired ratio, such as 1 in 10 to 1 in 3 radiopaque elements. Other ratios may also be implemented as disclosed herein.

In some embodiments, the interior volume 16 may comprise other forms and compositions beyond fiberfill, foam pieces, or fabric pieces. For example, the interior volume 16 may include beads, pellets, or particulate fill, such as plastic beads, glass beads, rubber pellets, or recycled materials. At least a portion of these beads or pellets may be manufactured from or coated with a radiopaque substance, such as barium-impregnated polyethylene, metal beads, or other high atomic number materials. The interior volume 16 may also comprise shredded textile scraps, natural fibers, wool, cotton batting, or blends thereof, with at least a portion of the scraps or fibers being radiopaque by virtue of impregnation, coating, or incorporation of radiopaque substances. In some embodiments, the interior volume 16 may include noise-emitting components, such as squeaker devices, crinkle paper, or plastic film, wherein at least a portion of these components comprises a radiopaque material. Additionally, the interior volume 16 may comprise gel or liquid fills containing radiopaque contrast agents, or natural radiopaque minerals such as small stones or shells. The radiopaque aspect of any of these components may be achieved by blending, coating, impregnating, or otherwise incorporating radiopaque substances, and the ratio of radiopaque to non-radiopaque elements may be selected to provide sufficient detectability by x-ray or other medical imaging modalities.

Each of the components of the trackable pet toy 10 and radiopaque materials 20 discussed following may be implemented alone, or in conjunction with each of the other described components.

In some embodiments as shown in FIG. 2, the trackable pet toy 10 may comprise the radiopaque material 20 as a radiopaque marker 20a affixed to the trackable pet toy 10 at a location where a portion 10a is likely to be chewed off by the pet and ingested. The radiopaque marker 20a may comprise any of metallic markers, tags, ties, buttons, cloth strips, clips and similar articles as disclosed herein that may comprise at least one radiopaque substance. The radiopaque marker 20a may further comprise tags, ties, buttons, and the like which are non metallic and having a high atomic number and/or density. The radiopaque marker 20a may be affixed to the external surface 14, disposed within the interior volume 16 or affixed to an internal surface of the interior volume 16 of the pet toy to form the trackable pet toy 10. The radiopaque markers 20a may be small components attached to various features on the toy which are likely to be ingested by a pet such that, upon ingestion, the likelihood that at least some of the radiopaque materials will be ingested is increased. Thus, the radiopaque materials may comprise radiopaque markers of small plates, beads, clips, ties, or tags affixed to the exterior of the pet toy or disposed within an interior volume of the toy.

In so doing, a pet toy can be made trackable and visible against the surrounding tissues during medical imaging procedures such as x-ray imaging, MRI, CT, x-ray fluoroscopy, ultrasound and radiography, as earlier disclosed. For example, a pet toy that comprises radiopaque materials 20 or radiopaque markers 20a is visible, or has enhanced visibility, during X-ray radiography (still images) or fluoroscopy (continuous real-time x-ray images) than a pet toy without radiopaque materials or radiopaque markers. Typically, a pet will detach and ingest a portion 10a of a pet toy, and when the ingested portion 10a of the trackable pet toy 10 comprises the radiopaque materials 20 and radiopaque markers 20a as disclosed herein, the ingested portion 10a may be tracked using medical imaging methods. A benefit provided by the ability to locate the ingested portion 10a within the pet's digestive system 32 (with numerous portions 10a as shown in FIG. 5) is the ability to monitor the movement of the ingested portion 10a over time to ensure the ingested portion 10a is passed without issue rather than becoming lodged in the digestive tract 32 as a blockage, which may require some form of intervention.

In some embodiments, the radiopaque markers 20a may comprise strips of cloth that are incorporated into the pet toy. For example, a radiopaque strip of cloth may be sewed to the pet toy at any location. The radiopaque strip of cloth may be colored to blend into the pet toy so as not to call attention to the radiopaque strip of cloth. Alternatively, the radiopaque strip of cloth may be colored to stand out and be noticeable on the pet toy to indicate to the pet owner the presence and location of the radiopaque strip, thus providing additional safety to the pet because the pet toy will be easier to locate within the pet's digestive system 32. In some embodiments, the radiopaque strip of cloth may be colored to blend into the pet toy, and an additional radiopaque marker may be included on the pet toy to indicate to the pet owner that the pet toy provides additional safety to the pet. As used herein, the terms “cloth” and “fabric” are meant to be interchangeable, and the use of one term versus the other is not intended to imply any difference.

Multiple radiopaque markers 20a comprising strips of cloth may be positioned on the pet toy to increase the likelihood that the pet ingests a portion of the pet toy comprising a radiopaque strip. In some embodiments, the radiopaque strips extend parallel to each other, while in other embodiments, the radiopaque strips extend along the pet toy parallel to some strips and perpendicular to others. This helps to increase the likelihood that, if a portion of the pet toy is ingested by the pet, then some radiopaque material will be ingested along with it. In embodiments with multiple radiopaque strips, a first portion of the radiopaque strips may be colored to blend in with the pet toy and a second portion of the radiopaque strips may be colored to stand out and indicate to the pet owner that the pet toy has radiopaque strips included. In some embodiments, the radiopaque strips may blend in, and in other embodiments, the radiopaque strips may stand out. Additionally, in some embodiments, rather than using color to call attention to the radiopaque strips, the strips may instead have a particular texture or shape to indicate the presence of the radiopaque strips to the pet owner.

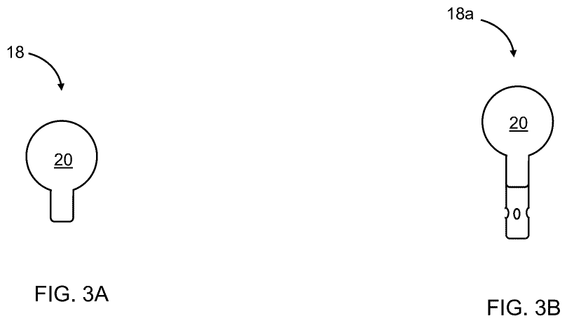

In some embodiments as shown in FIG. 2, the trackable pet toy 10 may comprise a noise emitting component 18, where the noise emitting component 18 comprises in part or in an entirety the radiopaque material 20. As shown in FIG. 2, the noise emitting component 18 may be disposed within interior volume 16. When comprised at least in part of the radiopaque materials 20 as disclosed herein, the noise emitting components 18 may be configured for detection using x-ray imaging within the digestive system 32 of an animal 30. According to exemplary embodiments as shown in FIGS. 3A and 3B, the noise emitting component 18 may comprise any of a squeak device, a plastic film, a crinkle paper, a silent ultrasonic squeaker device 18a, and combinations thereof, each comprising in part or in an entirety the radiopaque material 20. While the silent ultrasonic squeaker device (shown as 18a of FIG. 3B) is audible to a dog, a pet owner cannot hear sounds emitted from the silent ultrasonic squeaker device 18a. As such, a pet owner would not be alerted or know if a portion 10a of the toy which included the silent ultrasonic squeaker device 18a, or the silent ultrasonic squeaker device 18a itself, had been ingested by a pet. Thus, a trackable pet toy 10 comprising a silent ultrasonic squeaker device 18a made from the radiopaque materials 20 as disclosed herein may prove beneficial in determining whether the pet or dog has ingested the silent ultrasonic squeaker device 18a, and if it poses concern. The exemplary noise emitting components 18 as shown in FIGS. 3A and 3B may be disposed anywhere within the trackable pet toy 10, such as between the external surface 14 and the interior volume 16, or within the interior volume 16 inside the trackable pet toy 10, or in the interior volume 16 or pocket of the toy as shown in FIG. 1. According to some embodiments, the noise emitting component 18 may comprise a squeaker device. In an embodiment, the trackable pet toy may comprise a noise emitting component 18 comprised at least in part of polymeric radiopaque materials where the radiopaque materials 20 can be included in the pet toy according to the methods as disclosed herein.

### EXAMPLES

Example 1 (Radiopaque filament in shell). A plush dog toy may be constructed with a woven nylon shell having a thread count of approximately 200 threads/in², wherein about 1 in 50 warp threads comprised an x-ray detectable filament loaded with barium sulfate within an elastomeric sheath. After removal of a portion of the shell, the removed portion remains readily detectable on a veterinary x-ray image.

Example 2 (Radiopaque fiberfill blend). A stuffed toy may include a fiberfill blend comprising non-radiopaque polyester fibers and radiopaque fibers comprising a polymer loaded with tungsten and/or barium sulfate. The blend may include about 10% radiopaque fibers by count, providing sufficient visibility of torn stuffing fragments during x-ray imaging.

Example 3 (Coated fabric and marker pattern). A radiopaque polymer composition loaded with barium sulfate may be applied as a thin, flexible coating to selected seams and to a marker strip attached to an ear portion of the toy. The coating remains adhered after washing and chewing simulation, and the marker produces a distinctive signature on x-ray images to aid identification.

Example 4 (Discrete zone distribution and fragment detectability). A plush dog toy may be constructed with radiopaque regions located in four discrete exterior zones (two ears, a nose region, and a tail region) and radiopaque stuffing elements distributed throughout the interior volume. The radiopaque stuffing elements may comprise foam pieces having a characteristic dimension of about 2-8 mm, blended with non-radiopaque foam pieces at a ratio of about 10% radiopaque pieces by count. Even when the toy is torn into multiple fragments, a representative set of fragments (each fragment being at least about 2 cm\hat{ }2 of exterior fabric or at least about 5 cm\hat{ }3 of interior stuffing) each includes at least one radiopaque thread and/or at least one radiopaque stuffing element.

Example 5 (Qualitative detectability assessment). A sample toy constructed according to Example 1 and Example 2 may be radiographically imaged under standard veterinary radiography settings appropriate for a medium-size canine, with a soft-tissue-equivalent background. The radiopaque portions are visually distinguishable relative to the background and relative to adjacent non-radiopaque toy material. After being subject to a laundering cycle and a water rinse, a portion of the sample toy may be re-imaged. The radiopaque portions remain visually distinguishable, consistent with retention of the radiopaque material by the toy components.

The trackable pet toy 10 may be formed according to various methods of manufacturing. According to some embodiments, a method of manufacturing a trackable pet toy may comprise fabricating the pet toy from a woven fabric where the woven fabric comprises a radiopaque material comprising at least one radiopaque substance such that the pet toy becomes visible using medical imaging techniques. In a further embodiment, a method of manufacturing a trackable pet toy may comprise providing a pet toy manufactured from a woven fabric 12 and having an interior and an exterior, and attaching a radiopaque marker to at least a portion of the body of the pet toy to form a trackable pet toy, wherein the radiopaque marker is configured to increase visibility of the trackable pet toy when viewed using X-ray imaging. In a further embodiment, a method of manufacturing a trackable pet toy may comprise providing a trackable pet toy where at least a portion of the trackable pet toy is formed of a radiopaque material configured for detection using medical imaging within a digestive system of an animal. A yet further embodiment of a method of manufacturing a trackable pet toy may comprise providing a pet toy and providing a polymer solution comprising at least one radiopaque substance and applying the polymer solution to the pet toy such that upon curing, a trackable pet toy comprising a water insoluble, radiopaque polymer coating is formed. Methods of application may include submerging, dipping, or coating the pet toy with a polymer solution. The viscosity of the polymer solution may be adjusted according to the method of application. In a yet further embodiment, a method of manufacturing a trackable pet toy may comprise providing a trackable pet toy comprising a noise emitting component disposed therein, where the noise emitting component comprises a radiopaque material comprising at least one radiopaque substance.

In some embodiments, the principles described herein may be applied to objects that are not traditionally pet toys, but are nonetheless often played with by pets, such as hair ties that are commonly ingested by cats. A hair tie may comprise an elastic core, such as a rubber band, surrounded by a fabric shell. The fabric shell may be formed from woven or knitted threads, and at least one thread of the shell may comprise a radiopaque material. For example, a hair tie may include a thread containing barium sulfate or another radiopaque substance, which would render the hair tie detectable by x-ray imaging if ingested. The radiopaque thread may be indistinguishable by touch or sight from non-radiopaque threads, allowing for discrete implementation.

The radiopaque features described herein may be also applied to a variety of objects that are commonly ingested by pets, including but not limited to socks, underwear, washcloths, and other fabric-based items. By incorporating radiopaque threads, coatings, or markers into these objects, they may be rendered detectable by x-ray imaging or other medical imaging modalities if ingested by an animal. The radiopaque material may be incorporated into the fabric during manufacturing or applied as a coating or marker after fabrication.

It will be understood that implementations of the trackable pet toy include but are not limited to the specific components disclosed herein, as virtually any components consistent with the intended operation of various trackable pet toys may be utilized with other compatible components. Accordingly, for example, it should be understood that, while the drawings and accompanying text show and describe particular trackable pet toys, any such implementation may comprise any shape, size, style, type, model, version, class, grade, measurement, concentration, material, weight, quantity, and/or the like consistent with the intended operation of trackable pet toys.

The concepts disclosed herein are not limited to the specific trackable pet toys shown herein. For example, it is specifically contemplated that the components included trackable pet toys may be formed of any of many different types of materials or combinations that can readily be formed into shaped objects and that are consistent with the intended operation of the trackable pet toy. For example, the components may be formed of: rubbers (synthetic and/or natural) and/or other like materials; elastomers and/or other like materials; polymers such as thermoplastics (such as ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; high atomic number metals, such as titanium, copper, silver, zirconium, gold, platinum, tantalum, tungsten, gadolinium, iron, steel, carbon steel, alloy steel, tool steel, stainless steel, spring steel, and/or other like materials; and/or any combination of the foregoing including, without limitation, those materials common to pet toys in the pet toy industry.

Furthermore, trackable pet toy components may be manufactured separately and then assembled, or any or all the components may be manufactured simultaneously and integrally joined with one another. Manufacture of these components separately or simultaneously, as understood by those of ordinary skill in the art, may involve 3-D printing, extrusion, pultrusion, vacuum forming, injection molding, impregnation, immersion coating, dip coating, blow molding, resin transfer molding, casting, forging, cold rolling, milling, drilling, reaming, turning, grinding, stamping, sewing, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular trackable pet toys, it should be readily apparent that several modifications may be made without departing from the spirit thereof and that these implementations may be applied to other implementations disclosed or undisclosed. The presently disclosed trackable pet toys are, therefore, considered illustrative and not restrictive.

More specifically, this disclosure, its aspects and embodiments, are not limited to the specific material types, components, methods, or other examples disclosed herein. Many additional material types, components, methods, and procedures known in the art are contemplated for use with implementations from this disclosure. Accordingly, for example, although implementations are disclosed, such implementations and implementing components may comprise any components, models, types, materials, versions, quantities, and/or the like as is known in the art for such systems and implementing components, consistent with the intended operation.

What is claimed is:

1. A chewable pet toy, comprising:
a body defining an exterior layer and an interior volume; and
a radiopaque component retained by the body, wherein the radiopaque component is disposed in a portion less than all of at least one of the exterior layer or the interior volume and wherein the radiopaque component is detectable by radiographic imaging when located within a digestive tract of an animal.

2. The chewable pet toy of claim 1, wherein the radiopaque component comprises a radiopaque filament incorporated into a textile of the exterior layer.

3. The chewable pet toy of claim 2, wherein the textile comprises a woven fabric having a thread count, and wherein a fraction of radiopaque threads in the thread count is from 1 in 3 to 1 in 2000.

4. The chewable pet toy of claim 3, wherein the fraction of radiopaque threads is from 1 in 10 to 1 in 300.

5. The chewable pet toy of claim 1, wherein the radiopaque component comprises a radiopaque coating bonded to fibers of the exterior layer.

6. The chewable pet toy of claim 5, wherein the radiopaque coating comprises a polymer matrix loaded with a radiopaque filler at a loading of from 10 wt % to 70 wt %.

7. The chewable pet toy of claim 1, further comprising stuffing material disposed in the interior volume, wherein the stuffing material includes radiopaque stuffing elements and non-radiopaque stuffing elements.

8. The chewable pet toy of claim 7, wherein the radiopaque stuffing elements comprise from 0.5% to 50% of the stuffing elements by count.

9. The chewable pet toy of claim 7, wherein the radiopaque stuffing elements have a characteristic dimension from 1 mm to 20 mm, and wherein the pet toy comprises at least 25 radiopaque stuffing elements.

10. The chewable pet toy of claim 1, wherein the radiopaque component is distributed across a plurality of locations of the body including at least two spatially separated exterior zones.

11. A trackable pet toy, comprising:
a woven fabric forming an external surface of a body, the woven fabric having a thread count;
stuffing disposed within an interior volume of the body;
wherein a minority of the threads comprise radiopaque thread material;
wherein a minority of stuffing elements comprise radiopaque stuffing elements; and
wherein the radiopaque thread material and the radiopaque stuffing elements are water insoluble and retained by the trackable pet toy under washing and saliva exposure, such that the trackable pet toy is x-ray detectable after ingestion by an animal.

12. The trackable pet toy of claim 11, wherein the minority of the threads comprises from 1 in 5 to 1 in 500 threads of the thread count.

13. The trackable pet toy of claim 11, wherein the radiopaque thread material includes a core-sheath filament having a radiopaque core encapsulated by a non-radiopaque sheath.

14. The trackable pet toy of claim 11, wherein radiopaque material is disposed along from 10% to 80% of a total seam length of the woven fabric.

15. The trackable pet toy of claim 11, wherein radiopaque material is disposed in from 2 to 12 discrete exterior zones of the external surface.

16. The trackable pet toy of claim 11, wherein the radiopaque stuffing elements comprise foam pieces and/or fiberfill fibers including tungsten and/or barium sulfate.

17. The trackable pet toy of claim 11, wherein the radiopaque stuffing elements comprise from 1% to 30% of the stuffing elements by mass.

18. The trackable pet toy of claim 11, further comprising a noise-emitting component disposed within the interior volume, wherein at least a portion of the noise-emitting component is radiopaque.

19. A method of manufacturing a trackable pet toy, comprising:
incorporating a radiopaque filler into at least one of (i) threads used to form an exterior layer of a pet toy, (ii) stuffing elements used to fill an interior volume of the pet toy, or (iii) a polymeric coating applied to a textile component of the pet toy;
assembling the pet toy so that the radiopaque filler is retained by the pet toy and disposed in a portion less than all of at least one of the exterior layer or the interior volume; and
confirming radiographic detectability of the pet toy via radiographic imaging.

20. The method of claim 19, wherein incorporating the radiopaque filler comprises dispersing barium sulfate and/or tungsten in a polymer matrix and forming the polymer matrix into at least one of a filament, a foam piece, or a coating.
